(19) United States
(12) Patent Application Publication
(10) Pub. No.: US 2016/0000382 A1
(43) Pub. Date: Jan. 7, 2016

(54) SYSTEMS AND METHODS FOR EEG MONITORING

(71) Applicant: Cadwell Laboratories Inc., Kennewick, WA (US)

(72) Inventors: Jinesh J. Jain, Kennewick, WA (US); John Cadwell, Kennewick, WA (US); Alison Rhoades, Kennewick, WA (US)

(21) Appl. No.: 14/320,916

(22) Filed: Jul. 1, 2014

(51) Int. Cl. A61B 5/00; A61B 5/0482

(52) U.S. Cl. CPC A61B 5/7282; A61B 5/0482; A61B 5/7435; A61B 5/746; A61B 5/4094

(57) ABSTRACT

Systems, devices and methods are described for physiological monitoring, for example monitoring EEG signals to detect the onset or probability of adverse events. The systems, devices and methods discussed herein may monitor received EEG signals to identify trends or patterns in the signal that are either indicative of ongoing seizures or indicative of a future risk of seizure. The systems, devices and methods provide the user with increased control and flexibility in the monitoring processes that produce the alerts. In particular, in some implementations the physician is able to make adjustments during monitoring and customize the process by which EEG data is displayed and analyzed during the patient monitoring without pausing the monitoring to make the adjustments.

### BACKGROUND

EEG systems are used to monitor a patient's neurological state. The patient's brain waves are measured with the EEG systems and can be used for diagnosis, preventative treatment, or for monitoring patients during anesthesia, among other procedures. Such EEG systems include a number of sensors that are placed in contact with the patient's scalp, for example with a sensor cap. Each sensor detects electrical activity within the area of the brain beneath the scalp near the sensor, and trends or patterns within the detected electrical signals are used to make a diagnosis or determination regarding the patient's state. Multiple electrodes are placed in different locations along the scalp to detect signals from different hemispheres and different regions within the brain, and the different signals can be used make determinations regarding particular areas and functions of the brain.

In some applications, EEG signals are monitored to detect either the onset or probability of adverse neurological events. A monitoring system can detect such events by processing EEG signals to extract parameters that are indicative of the patient's neurological state. Patterns within those parameters known to be indicative of adverse effect, for example patterns indicative of a seizure, are tracked and monitored by the system to detect when an event has occurred or to warm of the risk of a future adverse effect. For example, sudden erratic variance in an EEG signal can signal that a patient is either experiencing or is about to experience a seizure.

Some trends and patterns in EEG signals that are indicative of adverse effects may be clear in raw EEG signal traces. For example, an EEG sensor signal changing suddenly from a smooth, flat signal to an erratic signal with multiple spikes can be seen right away either by a physician monitoring signals or automatically detected by the system that is processing the signals. In some cases, however, the indicators of adverse effects, for example indicators of seizure, are nuanced and based on multiple parameters spread across multiple EEG sensor channels. In these cases, it can be more difficult for a physician to pick out the points that are indicative of adverse events and to differentiate between an event that is close to an adverse effect and an actual adverse effect.

Automated EEG systems have been developed to automatically detect events rather than relying on a physician's judgment. These systems extract a variety of parameters from received EEG signals and apply an algorithm to determine whether or not an event is occurring or to calculate a probability that an event such as a seizure will occur in the near future. These systems typically apply a rigid pre-programmed algorithm to the extracted parameters to determine either a binary signal indicating whether or not an event is occurring or a scaled signal indicating the probability of an event. In some systems, the particular algorithm used may be tailored to individual patients or to a particular type of event detection. In these cases, a user can pre-program certain parameters used in the algorithm, either based on desired settings or based on identified adverse patterns from past signals. The pre-programmed algorithm is then used during patient monitoring. While these systems allow some variation in monitoring between different patients, the pre-programmed algorithm in these systems cannot be changed in the fly—i.e., while the physician is monitoring a patient. These systems do not provide a convenient and easy way for physicians to adjust and optimize the algorithm and monitoring system during ongoing patient monitoring.

### SUMMARY

Disclosed herein are systems, devices and methods for EEG monitoring and, in particular, for monitoring EEG signals to detect the onset or probability of adverse events. For example, the systems, devices and methods discussed herein may monitor received EEG signals to identify trends or patterns in the signal that are either indicative of ongoing seizures or indicative of a future risk of seizure. The approaches discussed provide automated systems and methods for monitoring the EEG signals and for alerting a physician or other medical professional when the monitored events or a risk of these events are detected. The systems, devices and methods provide the user with increased control and flexibility in the monitoring processes that produce the alerts. In particular, in some implementations the physician is able to make adjustments during monitoring and customize the process by which EEG data is displayed and analyzed during the patient monitoring without pausing the monitoring to make the adjustments.

In one aspect, a method for monitoring EEG signals includes receiving an EEG sensor signal, extracting a plurality of parameters from the received signal, and determining, with a processor, an event indicator from the plurality of extracted parameters. A display screen is generated, and the display screen includes the event indicator, the extracted plurality of parameters, and a user selectable option. The method includes receiving a user selection of the option in the display screen and, in response to the user selection, (1) updating the extracted plurality of parameters in the display screen and (2) updating an algorithm used to determine the event indicator.

In certain implementations, the display screen includes a threshold displayed with each of the plurality of extracted parameters, and the user selectable option is a request to change the displayed thresholds. The request to change the displayed thresholds may include one or more of a numerical value entry, a menu of selectable thresholds, and an adjustable threshold cursor displayed in the display screen. The display screen may also include an alert for each of the plurality of extracted parameters, with each alert indicating whether a corresponding extracted parameter exceeds a threshold.

In certain implementations, the display screen includes a user selectable menu of available parameters. The menu of available parameters may include only parameters that are not displayed on the display screen and not used in the event indicator determination, or the menu may include all parameters that can be extracted from the EEG sensor signal. If all parameters that can be extracted are displayed, the display screen includes a marker for a first set of extracted parameters that are used in the event indicator determination to differentiate the first set of extracted parameters from a second set of parameters that are not used in the event indicator determination.

A user selection from a menu of available parameters may be a selection of an unused parameter from the menu. In response to a user selection of the unused parameter, the method includes generating a graph of a trend for the unused parameter in the display and reprogramming the algorithm to include the unused parameter in the event indicator determination.

In certain implementations, the received signal comprises data from a plurality of EEG channels, and the display screen includes a user selectable menu of the EEG channels. A user selection from the menu of EEG channels may be a request to include or exclude an EEG channel during patient monitoring. When the request to include or exclude an EEG channel is received, the method includes graphing updated trends for the displayed parameters based on the inclusion or exclusion of the EEG channel and reprogramming the algorithm to include or exclude data for the selected EEG channel from at least one extracted parameter. The request may be a request to include or exclude data for the selected EEG channel from all of the extracted parameters, or the request may be a request to include or exclude data for the selected EEG channel from one of the extracted parameters without affecting EEG channel data for additional extracted parameters.

In certain implementations, the algorithm includes weighting factors associated with each of the plurality of extracted parameters. When weighting factors are programmed in the algorithm, the display screen may include user selectable options to change the weighting factors associated with displayed extracted parameters.

In certain implementations, the event indicator includes at least one of an alert that an event has happened, a warning that an event will happen, a percentage estimate of the chance an event will happen, and a binary indication of whether an event is currently happening. In some applications, the monitored event is a patient seizure, and the event indicator is a seizure indicator.

In one aspect, a system for monitoring EEG signals includes an EEG sensor and a monitor, and the monitor is configured to receive an EEG signal from the EEG sensor. The monitor also includes a processor configured to generate a display screen that includes an event indicator, a plurality of parameters extracted from the received signal, and a user-selectable option. The processor is configured to receive a user selection of the option in the display screen, and, in response to the user selection, (1) update the extracted plurality of parameters in the display screen and (2) update an algorithm used to determine the event indicator. In some applications, the processor is also configured to carry out any of the method steps described above in paragraphs [0006]-[0012].

In certain implementations, the monitor includes communications circuitry. The communications circuitry may be configured to transmit the generated display screen to a display device and receive the user selection of the option from the display device. The communications circuitry may also be configured to send commands to the EEG sensor.

In certain implementations, the monitor includes a user interface. The monitor may be configured to receive the user selection of the option from the user interface. The selected option may be an option that is displayed on a display device in communication with the monitor.

In one aspect, a system for monitoring EEG signals includes means for receiving an EEG sensor signal, means for extracting a plurality of parameters from the received signal, means for determining an event indicator from the plurality of extracted parameters, and means for generating a display screen that includes the event indicator, the extracted plurality of parameters, and a user selectable option. The system includes means for receiving a user selection of the option in the display screen, means for updating the extracted plurality of parameters in the display screen in response to the user selection, and means for updating an algorithm used to determine the event indicator in response to the user selection.

In certain implementations, the display screen includes a threshold displayed with each of the plurality of extracted parameters and user selectable options to change the displayed thresholds. The user selectable options may include one or more of a numerical value entry, a menu of selectable thresholds, and an adjustable threshold cursor. The display screen may also include an alert for each of the plurality of extracted parameters, with each alert indicating whether a corresponding extracted parameter exceeds a threshold.

In certain implementations, the display screen includes a user selectable menu of available parameters. The menu of available parameters may include only parameters that are not displayed on the display screen and not used in the event indicator determination, or may include all parameters that can be extracted from the EEG sensor signal. If all parameters that can be extracted are displayed, the display screen includes a marker for a first set of extracted parameters that are used in the event indicator determination to differentiate the first set of extracted parameters from a second set of parameters that are not used in the event indicator determination.

A user selection from a menu of available parameters may be a selection of an unused parameter from the menu. The system includes means for generating a graph of a trend for the unused parameter in the display when the unused parameter is selected. The system also includes means for reprogramming the algorithm to include the unused parameter in the event indicator determination in response to the selection.

In certain implementations, the received signal includes data from a plurality of EEG channels, and the display screen includes a user selectable menu of the EEG channels. The user selection of an option from the display screen is a request to include or exclude an EEG channel during patient monitoring. The system also includes means for graphing updated trends for the displayed parameters based on the inclusion or exclusion of the EEG channel and means for reprogramming the algorithm to include or exclude data for the selected EEG channel from at least one extracted parameter. The request may be a request to include or exclude data for the selected EEG channel from all of the extracted parameters, or the request may be a request to include or exclude data for the selected EEG channel from one of the extracted parameters without affecting EEG channel data for additional extracted parameters.

In certain implementations, the algorithm includes weighting factors associated with each of the plurality of extracted parameters. When weighting factors are programmed in the algorithm, the display screen includes user selectable options to change the weighting factors associated with the displayed extracted parameters.

In certain implementations, the event indicator includes at least one of an alert that an event has happened, a warning that an event will happen, a percentage estimate of the chance an event will happen, and a binary indication of whether an event is currently happening. In some applications, the monitored event is a patient seizure, and the event indicator is a seizure indicator.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout.

FIG. 1 shows an illustrative EEG event monitoring display.

FIG. 2 shows a block diagram of an EEG monitoring system.

FIG. 3 shows a flow diagram of a method of patient monitoring in which a user selects to remove a parameter from patient monitoring.

FIGS. 4 and 5 show updated EEG event monitoring displays after receiving a user selection to remove a parameter from patient monitoring.

FIG. 6 shows a flow diagram of a method of patient monitoring in which a user selects to add a parameter to patient monitoring.

FIGS. 7 and 8 show updated EEG event monitoring displays after receiving a user selection to add a parameter to patient monitoring.

FIG. 9 shows a flow diagram of a method of patient monitoring in which a user selects to adjust a parameter option.

FIGS. 10 and 11 show updated EEG event monitoring displays after receiving a user selection of an EEG parameter adjustment option.

FIG. 12 shows a flow diagram of a method of patient monitoring in which a user selects to remove an EEG channel from patient monitoring.

FIGS. 13 and 14 show updated EEG event monitoring displays after receiving a user to remove an EEG channel from patient monitoring.

### DETAILED DESCRIPTION

The systems, devices, and methods described below involve EEG monitoring for neurological events or risks. The systems receive EEG signals from patient sensors and extract parameters from those signals. An algorithm is applied to determine either if a monitored event is currently occurring or to determine a risk of the monitored event occurring in the near future. A variety of extracted parameters that are used for the event determination are discussed and are not limiting in this disclosure. Other parameters and other algorithm factors may be used without departing from the scope of this disclosure. In some applications, the approaches described herein monitor EEG signals for indications of seizure or seizure risk. Additional adverse effects or neurological events other than seizures may also be monitored using the systems, methods and approaches discussed herein. For example, EEG signals can be analyzed in connection with polysomnography studies, such as sleep staging or sleep apnea analyses. Such systems can utilize EEG signals, along with other physiological measures, to determine depth of sleep, occurrence of an apnea event, risk of an apnea event, or other relevant sleep events from the analyzed measurements.

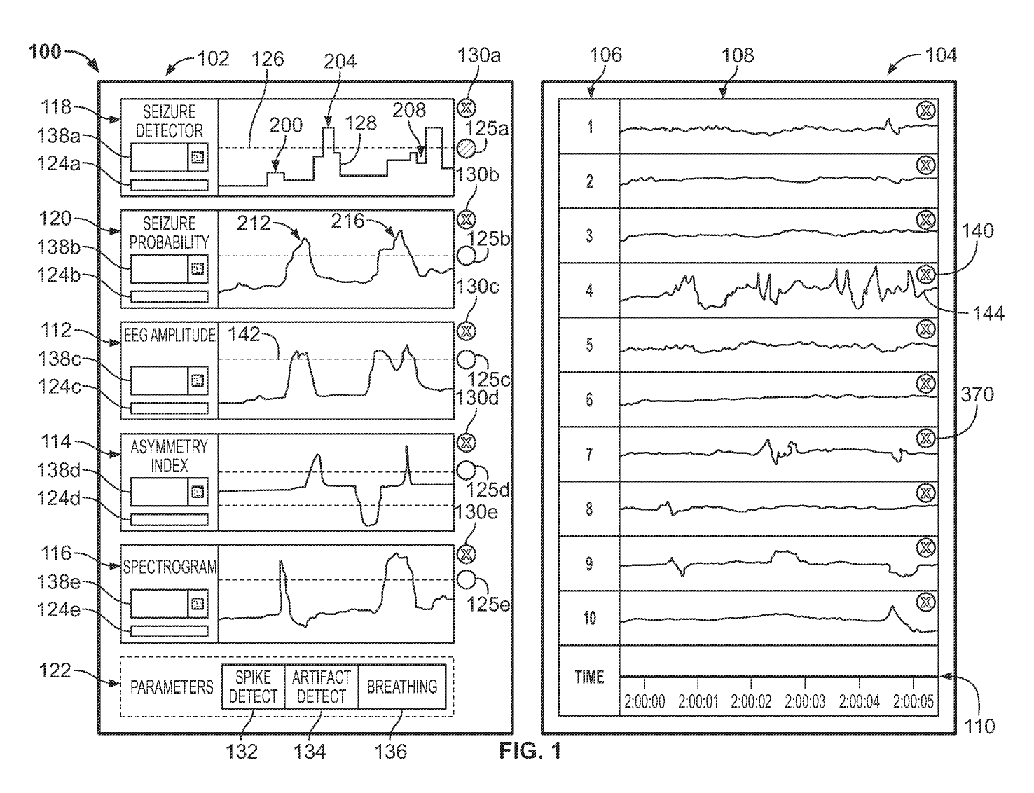

FIG. 1 shows an EEG monitoring and seizure detection system display 100. The display 100 includes two main windows, an EEG signal window 104 and a parameter window 102. The EEG signal window 104 shows a data trend for EEG signal channels. The EEG signals in the window 104 are identified by an identifier column 106 that indicates to a physician the EEG channel sensor associated with the particular signal. Window 104 also includes a signal column 108 that shows the time trend of each received EEG channel, and the bottom of window 104 displays a time line 110 that indicates the timing of each signal in the signal column 108.

The parameter window 102 displays for a physician or other user a plurality of parameters that are extracted from the EEG signals displayed in the signal window 108. The parameter window 102 also displays the calculated output of an event or seizure detection algorithm. In particular, the window 102 shows an indication that a seizure or event is occurring or shows the probability that an event will occur in the near future. In some embodiments, both a binary alert indicating whether or not an event is occurring and a probability calculation indicating a risk of a future event occurrence are included in the window 102. The parameters shown in the window 102 include an EEG amplitude trend 112, an asymmetry index trend 114, and a spectrogram trend 116. These three parameters are illustrative, and any number of suitable parameters may be shown in the window 102, including additional parameters that are extracted or determined from the EEG signals in window 108, or from other signals that are received by the monitoring system. The parameters 112, 114 and 116 are processed by the monitoring system to determine a seizure detector output that is shown in trend 118, and a seizure probability output that is shown in trend 120. Additional parameters that are not displayed in the window 102 may be used in the algorithm, depending on the number of parameters used and the space available on the display device on which the window 102 is presented.

The seizure detector trend 118 indicates whether or not a monitored patient is currently experiencing a seizure. The detector trend 118 is a binary alert that tells a physician or other user whether or not the monitored event is currently indicated in the EEG signals detected from a patient. The detector trend 118 usually presents the results of analyzing the parameters 112, 114 and 116, as well as any other monitored parameters, to determine whether or not each parameter exceeds a threshold or a patient for that parameter. The detector trend 118 indicates the aggregate number of parameters that exceed their respective thresholds, and when the number of parameters exceeding their individual thresholds is above a certain value, the trend 118 alerts the physician that a seizure is occurring. The alert may be a visual alert, for example a red light in indicator 1249, an audible alert, or both.

The seizure probability trend 120 is an algorithm output that combines the parameters 112, 114 and 116, as well as any other parameters monitored by the system, to determine the probability of a seizure occurring within a set future time window. The underlying algorithm used to calculate the probability trend 120 may be any suitable combination of the extracted parameters, including linear and non-linear weighted combinations of the parameters or any other calculations suitable for determining the probability of an oncoming event. The particular algorithm used to determine the probability trend 120 and the combination of the parameters used to determine the seizure detector trend 118 can be configured by the physician both before and during patient monitoring directly from the display 100.

The windows in display 100 analyze EEG signals to determine the occurrence or risk of seizure, but the present disclosure is not limited to only seizure detection. EEG analysis is utilized, either alone or in conjunction with other physiological measurements, to determine a wide range of neurological states, and the prediction or detection of other types of events can be achieved using the systems and methods discussed herein. For example, EEG signals can be analyzed to determine other types of adverse neurological effects or neurological states, such as depth of consciousness. EEG signals may also be used in polysomnography, either alone or in combination with other physiological measures, to analyze a patient's sleep. The EEG signals can be indicative of sleep stages or apnea detection, and the systems and methods described herein can be employed to similarly facilitate customization of the algorithms by which that analysis is carried out.

The two windows 102 and 104 in the display 100 provide a physician with improved flexibility to control the EEG monitoring information displayed in the display 100. Different patients and different monitoring environments may be more effective with varied combinations of parameters or different weightings or thresholds for the parameters in order to obtain accurate results in the seizure detector trend 118 and seizure probability trend 120. The display 100 provides the physician with control over the algorithms used to determine the trends 118 and 120, as well as the displayed information. The physician may use this control during ongoing monitoring to change parameters or parameter settings and select EEG channels or channel settings in order to improve the accuracy of the ongoing patient monitoring.

The display 100 shows the running trends of the different monitored EEG channels in the signal window 108, while also indicating to the physician the various parameters and event detection outputs in the window 102. To facilitate the physician's interpretation of these parameters, each of the trends displayed in the window 102 includes one of indicators 124a-e, that provide the physician with a quick visual alert that indicates whether or not a respective parameter or trend is exceeding a set threshold. For example, the seizure detector trend 118 includes an alert 124a that tells a physician whether or not the traced detector trend 128 is exceeding the threshold 126 set for that detector. When the trend 128 exceeds the threshold 126, this indicates that the number of extracted parameters from the EEG signals that meet or exceed their corresponding set thresholds is enough to indicate that the monitored patient is currently experiencing a seizure. The indicator 124a can be a color indicator that changes color whenever the trend 128 crosses the threshold 126 to alert the physician either to the onset of a seizure or to the end of a seizure. For example, when the trend 128 passes the threshold 126, the indicator 124a may change from a safe color, such as green, to a set seizure color, such as orange or red. The indicator 124a remains red until the trend 128 falls back below the threshold 126, at which time the indicator 124a switches back to the safe non-seizure color. The signal window 108 may also include circular alerts 125a-e in addition to or instead of the indicators 124a-124e. The circular alerts provide indications of the status of particular measurements, for example by displaying a certain color or by displaying a shading, like alert 125a in FIG. 1, or a blank alert, like alerts 125b-e in FIG. 1.

The patient monitoring control and flexibility provided by the display 100 allows a physician to manipulate the parameters that are displayed in the window 102 and that are used in the algorithms that determine the trends 118 and 120. For example, a parameter menu 122, included in the window 102, lists three parameters, a spike detect 132, an artifact detect 134, and a breathing parameter 136. Each of these parameters 132, 134 and 136 can be selected by the physician to add to the monitoring algorithm used to determine the detector trend 118 and the probability trend 120. The selection of a parameter from menus 122 may also cause the monitoring system to add the selected parameter to the window 102 displayed to the physician. The menu 122 may include only parameters that are not currently being used in the monitoring algorithms, or may include both parameters that are and are not included in the algorithms. If both parameters that are and parameters that are not currently used are in the menu 122, a shading or other marker may be placed on the parameters that are used in the algorithm but are not displayed on the window 102 to differentiate them from parameters that are not included in the monitoring algorithms at all. If a parameter that is not in the current algorithm is selected from the menu 122, the monitoring system may update the algorithm to factor in that selected parameter in determining the detector trend 118 and the probability trend 120. Additionally, the selected parameter may be added to the trends shown in the window 102. If a parameter that is already used in the algorithm but not currently displayed in the window 102 is selected from the menu 122, the system may update the display 100 to add the selected parameter to the window 102. The new parameter trend may be added in addition to the parameters already displayed, which may require scaling the size of those windows, or may replace one of the displayed trends. These adjustments to the display 102 and the monitoring algorithm are done “on the fly” during monitoring allowing the user to adjust the ongoing monitoring process without pausing.

A physician may also elect to remove one of the parameters from monitoring using the remove options 130a-e that are shown for each parameter and output window in the window 102. To remove one of the parameters 112, 114 and 116 from the monitoring algorithm and from the displayed window 102, a physician selects one of the respective close options 130c, 130d and 130e. When the physician selects one of these options, the corresponding parameter is removed from the window 102, and the data relating to that parameter is removed from the underlying algorithm that determines the trends 118 and 120.

The physician can also adjust the parameters and monitoring algorithm without adding or removing parameters completely from the underlying data processing. Each of the trends shown in window 102 includes a user selectable option box 138a-e that allows the physician to adjust the settings for the particular parameter shown in the corresponding trend. The adjustment made in response to the selection of one of options 138a-e depends on the particular option chosen or the particular parameter shown in the corresponding trend. For example, the option 138c for the EEG amplitude parameter 112 allows the user to adjust the threshold 142 that is applied to the parameter 112. For this adjustment, the option 138c may be a dropdown menu of possible changes in the threshold 142 or may be a window that indicates the numerical value of the threshold 142 and allows the physician to change and enter a new numerical value for the threshold 142. In some implementations, the option 138c allows the physician to select particular EEG channels, such as one or more of the channels shown in window 104, which are used to determine the amplitude parameter 112 and processed by the algorithms that determine the output trends 118 and 120. In some implementations, the option 138c is a dropdown menu that allows the user to select corresponding channels from the channel column 106 to either add or remove from the data that is processed and used to determine the amplitude parameter 112.

The EEG signal window 104, like the parameter window 102, of the display 100 may also provide the physician with options to change and adjust the patient monitoring algorithm and process on the fly without pausing monitoring. Each of the EEG trends shown in the signal column 108 includes a remove option, such as the option 140 shown for channel four. A physician may select to remove the data associated with that channel from patient monitoring and the determination of trends 118 and 120 of the parameter window 102. The remove option is helpful if a physician determines that data coming from one of the EEG sensors applied to the patient is not reliable, for example due to excess noise. In this case, the physician selects the remove option for that sensor to take the corresponding noisy data out of the patient monitoring processing and improve the accuracy of the monitoring output. If the physician determines, for example, that the data shown in the trend 144 of EEG channel four is an irregular signal that indicates excess noise and not actual patient data, the physician can select the remove option 140. The selection of option 140 takes the EEG channel four, and the data received from the corresponding sensor, out of the patient monitoring routine and reduces the chance of noise disrupting the monitoring outputs. If the physician selects the remove option 140, the algorithm used to run the patient monitoring process is updated to remove the data received from the sensor corresponding to channel four, and the trend 144 is removed from the signal column 108 in the window 104. The trend 144 may either be replaced with a different channel that is currently being monitored or may be removed from the column 108 without adding another channel.

In addition to the remove option 140, the window 104 may include a menu or a dropdown list that allows the user to select individual channels to either be included in the column 108 or the underlying data processing algorithm used to determine the outputs 118 or 120. The menu may also allow a user to make a single selection to both add a trend for a selected channel in the column 108 and add data received from the sensor corresponding to that channel to the data processing algorithm. Such a dropdown menu may include tick boxes that allow a user to individually check or uncheck the channels shown in the column 106, and any other EEG channels not currently shown in the window 104, to select the EEG channels displayed and used for monitoring.

The user selectable options presented in the display 100, for example the remove parameter options 130a-e, the parameter adjustment options 138a-e, parameter menu 122, and the remove EEG channel option 140, provide a physician with control and flexibility to adjust patient monitoring on the fly. These options streamline adjustment and reconfiguration of patient monitoring by each giving the physician selectable options that can both change the trends displayed in the display 100 and adjust the underlying algorithms used to determine the monitoring output trends 118 and 120 in the display 100. For example, by making a single selection of one of the remove options, such as parameter remove option 130c or EEG channel remove option 140, the physician removes the selected parameter or channel from the display 100 and also adjusts the algorithm used to determine the seizure detector trend 118 and the seizure probability trend 120 to account for the requested removal. Likewise, a selection from the menu 122 causes a system to both update the window 102 to include the selected parameter and reprogram the underlying algorithm for the output trends 118 and 120 to include the physician's desired parameter. This flexibility provides improved monitoring as the physician can make changes and adjustments on the fly and observe the changes in the monitoring that result from his selections in real time. Previous systems that allow the physician to adjust monitoring algorithms require the physician to preprogram algorithms used to determine neuromonitoring outputs and then begin patient monitoring. The inclusion of these options directly in the monitoring display 100 streamlines the process for the physician, and facilitates quicker changes and quicker optimization, as the physician can view the effects of selected changes live as patient data is continuously monitored.

The systems and methods described herein employ computer-implemented data processing to automate neurological event detection. The computer devices process data using programmed algorithms to detect the desired monitoring features in EEG signals, rather than requiring manual identification of these patterns in ongoing EEG signals by a physician. Various implementations of devices that are usable for the methods and patient monitoring described above for detecting neurological events are envisioned, including general programmable patient monitoring devices and processing systems as well as EEG-specific monitoring devices. For ease of illustration, an embodiment of these devices is described below with respect to an illustrative computing device. The systems, devices, and methods disclosed herein, however, may be adapted to other implementations and other embodiments of such devices.

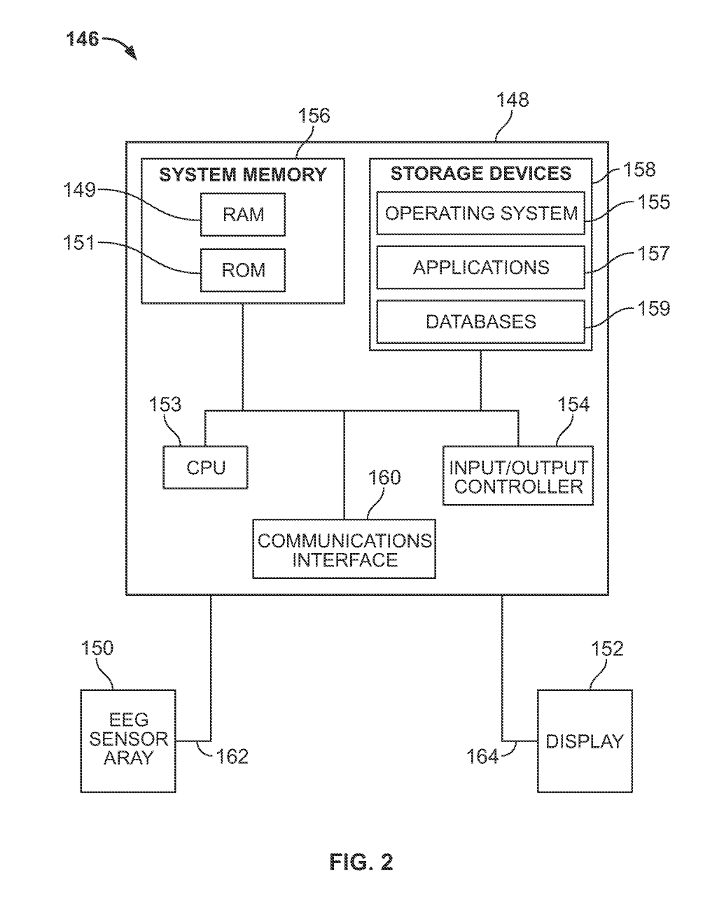

As used herein, the terms “processor,” “processing circuitry,” or “computing device” refers, without limitation, to one or more computers, microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores) or supercomputer. It may also refer to other devices configured with hardware, firmware, and software to carry out one or more of the computerized techniques described herein. Processors and processing devices may also include one or more memory devices for storing inputs, outputs, and data that is currently being processed. An illustrative computing device, which may be used to implement any of the processing circuitry and servers described herein, is described in detail below with reference to FIG. 2.

As used herein, “user interface” includes, without limitation, any suitable combination of one or more input devices (e.g., keypads, a mouse, touch screens, trackballs, voice recognition systems, gesture recognition systems, accelerometers, RFID and wireless sensors, optical sensors, solid-state compasses, gyroscopes, stylus input, joystick, etc.) and/or one or more output devices (e.g., visual displays, speakers, tactile displays, printing devices, etc.) For example, user interfaces can include a display (which may be a touch-sensitive color display, optical projection system, or other display) for graphically receiving and providing information to the user.

FIG. 2 shows a block diagram of an illustrative computer monitoring system 146 for detecting, diagnosing, or predicting neurological events from EEG signals. The system includes a computing device 148, EEG sensor array 150, and a display device 152. During monitoring, the EEG sensor array 150 detects electrical signals from a patient and communicates those signals over communications link 162 to the computing device 148. The communications link 162 may be a wired or wireless link, depending on the type of monitor and EEG sensor used. Computing device 148 processes the received signals, extracts parameters that characterize the EEG data, and generates a display of the data for a user. The generated display is transmitted from the computing device 148 to the display device 152 over communications link 164, which may also be a wired or wireless communications link depending on the devices used. In some implementations, the display device 152 is incorporated into the computing device 148, for example as a display screen on a patient monitor. The physician makes adjustments and selections from the screens displayed on the display device 152, and those adjustments are communicated back to the computing device 148 over the communications link 164. The selection is then processed by the computing device 148 to update the monitoring algorithm used to process data and to update the generated display screens that are transmitted to the display device 152. In some implementations, the selection may also affect the function of the EEG sensor array 150, and the computing device 148 transmits commands to the array 150 over the communications link 162 in response to the selection.

The computing device 148 includes at least one communications interface 160, an input/output controller 154, system memory 156, and one or more data storage devices 158. The system memory 156 includes at least one random access memory (RAM 149) and at least one read-only memory (ROM 151). These elements are in communication with a central processing unit (CPU 153) to facilitate the operation of the computing device 148.

The computing device 148 may be configured in many different ways. For example, the computing device 148 may be a conventional standalone computer or alternatively, the functions of computing device 148 may be distributed across multiple computer system and architectures. In FIG. 2, the computing device 148 is linked, via wireless or wired communications links 162 and 164, to sensor array 150 and display device 152. The computing device 148 may be configured in a distributed architecture, wherein databases and processing circuitry is housed in separate units or locations. Some units perform primary processing functions and contain at a minimum a general controller or a processing circuitry and a system memory. In distributed architecture implementations, each of these units may be attached via the communications interface 160 to a communications hub or port (not shown) that serves as a primary communication link with other servers, client or user computers and other related devices. The communications hub or port may have minimal processing capability itself, serving primarily as a communications router. A variety of communications protocols may be part of the system, including, but not limited to, Ethernet, SAP, SAS™, ATP, BLUETOOTH™, GSM, DICOM and TCP/IP.

Communications interface 160 is any suitable combination of hardware, firmware, or software for exchanging information with external devices. Communications interface 160 may exchange information with external systems using one or more of a cable modem, an integrated services digital network (ISDN) modem, a digital subscriber line (DSL) modem, a telephone modem, an Ethernet card, or a wireless modem for communications with other devices, or any other suitable communications interface. In addition, the communications interface 160 may include circuitry that enables peer-to-peer communication, or communication between user devices in locations remote from each other.

The CPU 153 includes a processor, such as one or more conventional microprocessors and one or more supplementary co-processors such as math co-processors for offloading workload from the CPU 153. The CPU 153 is in communication with the communications interface 160 and the input/output controller 154, through which the CPU 153 communicates with other devices such as other servers, user terminals, or devices. The communications interface 160 and the input/output controller 154 may include multiple communication channels for simultaneous communication with, for example, other processors, servers or client terminals.

The CPU 153 is also in communication with the data storage device 158 and system memory 156. The data storage device 158 and system memory 156 may comprise an appropriate combination of magnetic, optical or semiconductor memory, and may include, for example, RAM 149, ROM 151, flash drive, an optical disc such as a compact disc or a hard disk or drive. The system memory 156 may be any suitable combination of fixed and/or removable memory, and may include any suitable combination of volatile or non-volatile storage. The memory 156 may be physically located inside a monitoring device or may be physically located outside of the monitoring device (e.g., as part of cloud-based storage) and accessed by the monitoring device over a communications network. The CPU 153 and the data storage device 158 each may be, for example, located entirely within a single computer or other computing device; or connected to each other by a communication medium, such as a USB port, serial port cable, a coaxial cable, an Ethernet cable, a telephone line, a radio frequency transceiver or other similar wireless or wired medium or combination of the foregoing. For example, the CPU 153 may be connected to the data storage device 158 via the communications interface 160. The CPU 153 may be configured to perform one or more particular processing functions.

The data storage device 158 may store, for example, (i) an operating system 155 for the computing device 148; (ii) one or more applications 157 (e.g., computer program code or a computer program product) adapted to direct the CPU 153 in accordance with the systems and methods described here, and particularly in accordance with the processes described in detail with regard to the CPU 153; and/or (iii) database(s) 159 adapted to store information that may be utilized by the program.

The operating system 155 and applications 157 may be stored, for example, in a compressed, an uncompiled and an encrypted format, and may include computer program code. The instructions of the program may be read into a main memory of the processing circuitry from a computer-readable medium other than the data storage device, such as from the ROM 151 or from the RAM 149. While execution of sequences of instructions in the program causes the CPU 153 to perform the process steps described herein, hard-wired circuitry may be used in place of, or in combination with, software instructions for implementation of the processes of systems and methods described in this application. Thus, the systems and methods described are not limited to any specific combination of hardware and software.

Suitable computer program code may be provided for performing one or more functions in relation to aligning dietary behavior as described herein. The program also may include program elements such as an operating system 155, a database management system and “device drivers” that allow the processing circuitry to interface with a user interface or computer peripheral devices (e.g., a video display, a keyboard, a computer mouse, etc.) via the input/output controller 154.

The term “computer-readable medium” as used herein refers to any non-transitory medium that provides or participates in providing instructions to the processing circuitry of the computing device 148 (or any other processing circuitry of a device described herein) for execution. Such a medium may take many forms, including but not limited to, non-volatile media and volatile media. Non-volatile media include, for example, optical, magnetic, or opto-magnetic disks, or integrated circuit memory, such as flash memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM or EEPROM (electronically erasable programmable read-only memory), a FLASH-EEPROM, any other memory chip or cartridge, or any other non-transitory medium from which a computer can read.

Various forms of computer readable media may be involved in carrying one or more sequences of one or more instructions to the CPU 153 (or any other processing circuitry of a device described herein) for execution. For example, the instructions may initially be borne on a magnetic disk of a remote computer (not shown). The remote computer can load the instructions into its dynamic memory and send the instructions over an Ethernet connection, cable line, or even telephone line using a modem. A communications device local to a computing device 148 (e.g., a server) can receive the data on the respective communications line and place the data on a system bus for the processor. The system bus carries the data to main memory, from which the processing circuitry retrieves and executes the instructions. The instructions received by main memory may optionally be stored in memory either before or after execution by the processor. In addition, instructions may be received via a communication port as electrical, electromagnetic or optical signals, which are exemplary forms of wireless communications or data streams that carry various types of information. The combination of processing power and programmable logic in the computing device 148 provides a system that automates the monitoring procedure while still allowing sufficient control for a physician to improve the monitoring routines used to analyze a patient's EEG signals.

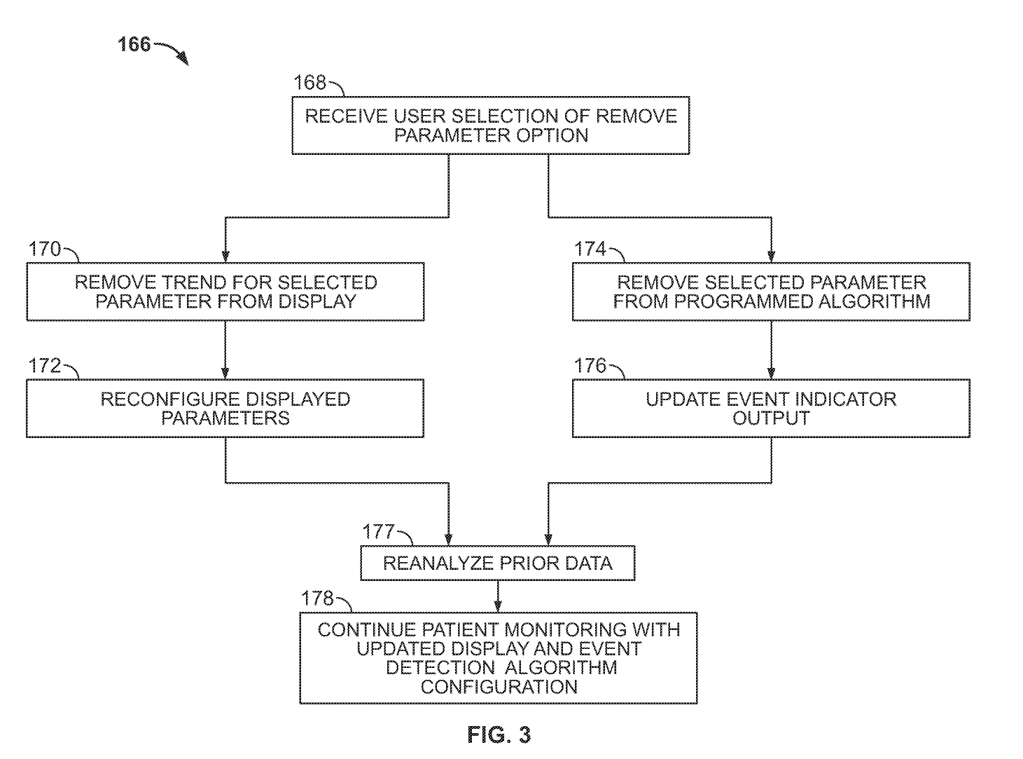

The flexibility and improved monitoring provided to a physician from the display screen shown in FIG. 1 implemented with a monitoring system such as the monitoring system shown in FIG. 2 is described below with respect to several illustrative and non-limiting examples of options provided to the physician. FIGS. 3-5 show a method and corresponding display screens that facilitate a user's removal of an extracted EEG signal parameter from monitoring while patient monitoring is ongoing. In the flowchart 166 shown in FIG. 3, the process begins when a user selection of an option to remove a parameter is received at step 168. This received selection may correspond, for example, to a selection of the remove parameter option 130d in the display screen 100 of FIG. 1. The physician selects the remove option 130d to take the asymmetry index parameter 114 out of the ongoing patient monitoring routine. Returning to FIG. 3, in response to receiving that selection at step 168, the monitoring system performs two parallel updates. The first update adjusts a display screen such as the screen 100 to account for the removed parameter, and a second update reprograms the underlying monitoring algorithm that determines the event detection and probability outputs of the monitoring system. The system may also optionally reanalyze past data and update the event detector output for past measurements to account for the adjustments.

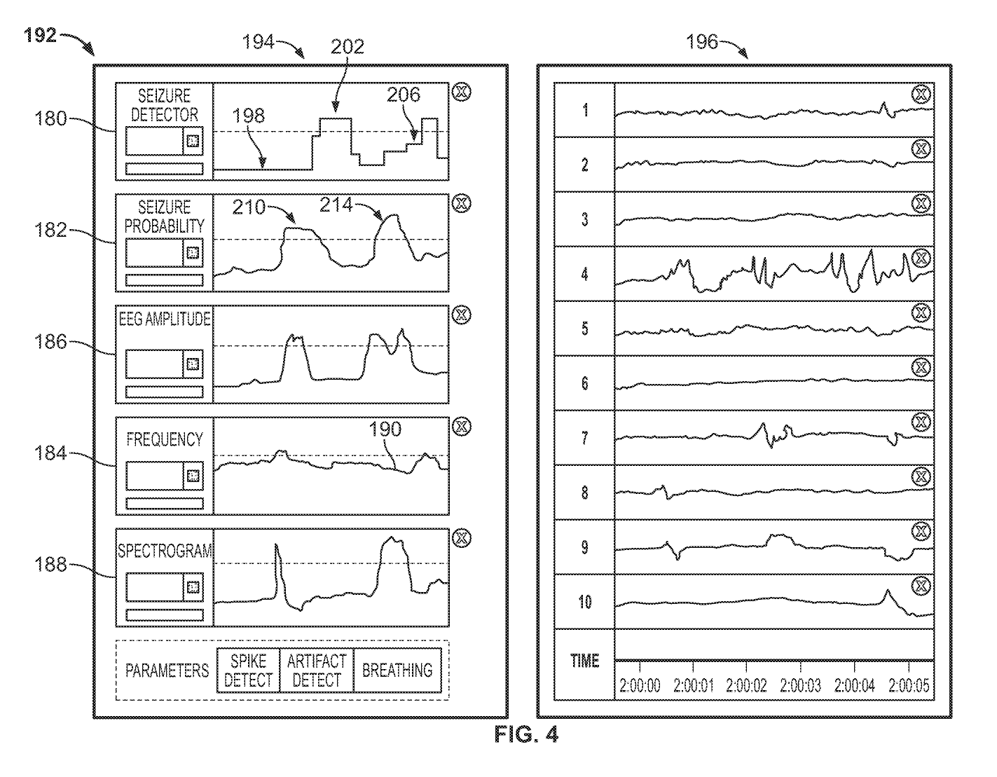

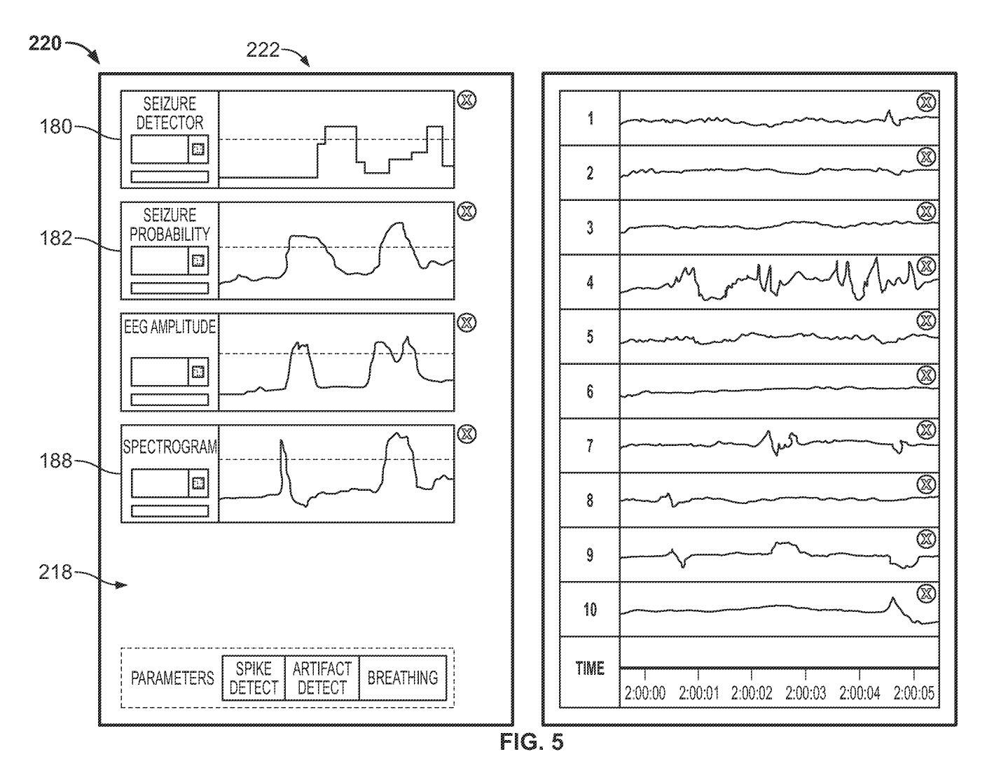

The first of the updates, regenerating the display screen, begins at step 170 in which the trend for the parameter indicated in the user selection is removed from the display. For the example in which remove option 130d is selected from the screen 100 in FIG. 1, the corresponding parameter is the asymmetry index parameter 114. At step 170 in FIG. 3, the asymmetry index parameter would be removed from the display presented to the physician. In the second step of updating the display, the remaining displayed parameters are reconfigured at step 172. This reconfiguration, as shown in FIGS. 4 and 5, may include scaling the remaining parameters, rearranging the remaining parameters or adding one or more new parameters to the display to replace the removed trend.

In response to the user selection received at step 168, the programmed monitoring algorithm is updated in parallel with the display update at steps 174 and 176. At step 174, the selected parameter, in particular data corresponding to that parameter, is removed from the programmed algorithm. This step may include, for example, weighting the corresponding parameter to a zero weight in the algorithm, such that any data for that parameter extracted from the received EEG data is cancelled out of the algorithm calculation. In addition to removing the selected parameter at step 174, other parameters in the algorithm may be adjusted and reweighted if necessary to account for the removed parameter based on the type of algorithm used in the calculation. At step 176, the event indicator, for example the seizure detector trend 118 or the seizure probability trend 120 in FIG. 1, is updated to account for the change in the reprogrammed monitoring algorithm. In some implementations, updating the event indicator includes recalculating the data for the event indicator using the data trends that are already displayed in the display screen. This is performed at step 177, in which the prior data is reanalyzed to take into account the change requested by the user. The update causes the graphed trends for past data to update and retroactively show recalculated event indicator trends. In other implementations, the update changes only event indicators forward from the time that the removed parameter option is selected at step 168. In these implementations, step 177 is bypassed, and the prior data trends remain, while the user's adjustment takes effect only going forward.

After both the display and the underlying algorithm are updated, patient monitoring continues at step 178. The ongoing patient monitoring incorporates both the display updates from steps 170 and 172 and the reprogrammed algorithm and event indicator updates from steps 174 and 176, thus providing a revised monitoring configuration at step 178 in response to the single selection from the user that is received at step 168.

The method shown in FIG. 3 illustrates a process by which the systems and methods described herein to update the physician's display and reprogram the underlying monitoring algorithm from a single physician selection. The display screens in FIGS. 4 and 5 show effects of updating both the display and underlying algorithm on the ongoing monitoring after the physician selection without stopping patient monitoring. In FIG. 4, a display screen 192 includes two windows, a parameter window 194 and an EEG sensor signal window 196, that illustrate changes resulting from the method shown in FIG. 3. As shown in the parameter window 194, the asymmetry index parameter selected by the user has been removed from the displayed parameters. In place of the asymmetry parameter, a new frequency parameter 184, with a trend 190 illustrating the frequency of EEG signals over the monitored time window, has replaced the asymmetry index trend. Both the EEG amplitude trend 186 and the spectrogram trend 188 remain unchanged from the display screen 100 in FIG. 1, as neither of these trends is affected by the inclusion or exclusion of the independent asymmetry index parameter in the algorithm. Thus, the physician's selection to remove the asymmetry index from monitoring does not have any affect on the amplitude or spectrogram parameters that are extracted from the raw EEG data shown in the EEG sensor window 196.

In contrast to the parameters 186 and 188, the two event indicators, seizure detector trend 180 and seizure probability trend 182, are changed in the parameter window 194 relative to the parameter window 102 of FIG. 1. The change in the displayed trends 180 and 182 is the result of the dual effect of the selection of the user remove parameter option 130d in FIG. 1. The display window 194 is updated to remove the selected asymmetry index parameter corresponding to the option 130d, and the monitoring algorithms that determine the detector trend 180 and probability trend 182 are also affected, thus changing some portions of the displayed trends 180 and 182. The parameter window 192 shows changes retroactively for data already processed before option 130d is selected in the trends 180 and 182 to highlight the effect of the selection of the option 130d. In some embodiments, the systems and methods described herein may not retroactively change the displays, but rather only update the algorithm and displays going forward from the point at which the option is selected.

In the seizure detector trend 180, there is a flat portion 198 at the beginning of the trend that corresponds to the plateau 200 from the corresponding display of the detector trend 118 in FIG. 1. The flat portion 198 does not increase and does not have a plateau as a result of the change in the parameters that are considered in the underlying algorithm that determines the detector. For the purpose of illustration, as a result of the removal of the asymmetry index from the monitoring algorithm, there is no longer a mark in the detector trend at the portion 198. Likewise, the shape of the data trend at portions 202 and 206 of the new updated seizure detector trend 180 are slightly different than the corresponding previous sections 204 and 208 of the detector trend 118. Again, these changes in the detector output is the result of the removal of the asymmetry index from the monitoring algorithm. Like the seizure detector trend 180, the seizure probability trend 182 is changed from the corresponding trend 120 of FIG. 1. In particular, peaks 210 and 214 in the probability trend 182 are slightly different in shape than corresponding peaks 212 and 216 in the original trend 120.

As an alternative to adding the new frequency parameter 184 to the display 192 when the user selection is received, the removal of the asymmetry index may leave a blank spot in the parameter window 194. FIG. 5 shows an updated display 220 after removal of the asymmetry index, including a parameter window 222. In the parameter window 222, the asymmetry index trend has been removed, and the spectrogram parameter 188 has been shifted upwards to replace the area left by the asymmetry index. The move of the spectrogram parameter 188 leaves a blank area 218 on the parameter window 222. The menu of available parameters may also be moved up below the spectrogram parameter 188, leaving the blank space 218 at the bottom of the window, rather than between the spectrogram trend and the menu. In other implementations, the remaining parameters may all be scaled to take up the blank area 218 rather than leaving a gap in the window. The user may then be given an option to select a new parameter for display, which may either be a parameter currently being monitored or a parameter not currently being monitored to fill, the area 218. For example, when the asymmetry index is removed and the spectrogram parameter 188 is moved up, the system may automatically display an option to the user listing the other parameters currently being used in the monitoring algorithm, but not yet displayed in the parameter window 222. The user may select from among these monitored parameters to fill the area 218. Also in the display 220, as with the display 192, the seizure detector trend and seizure probability trend 180 and 182 are updated to account for the removal of the asymmetry index.

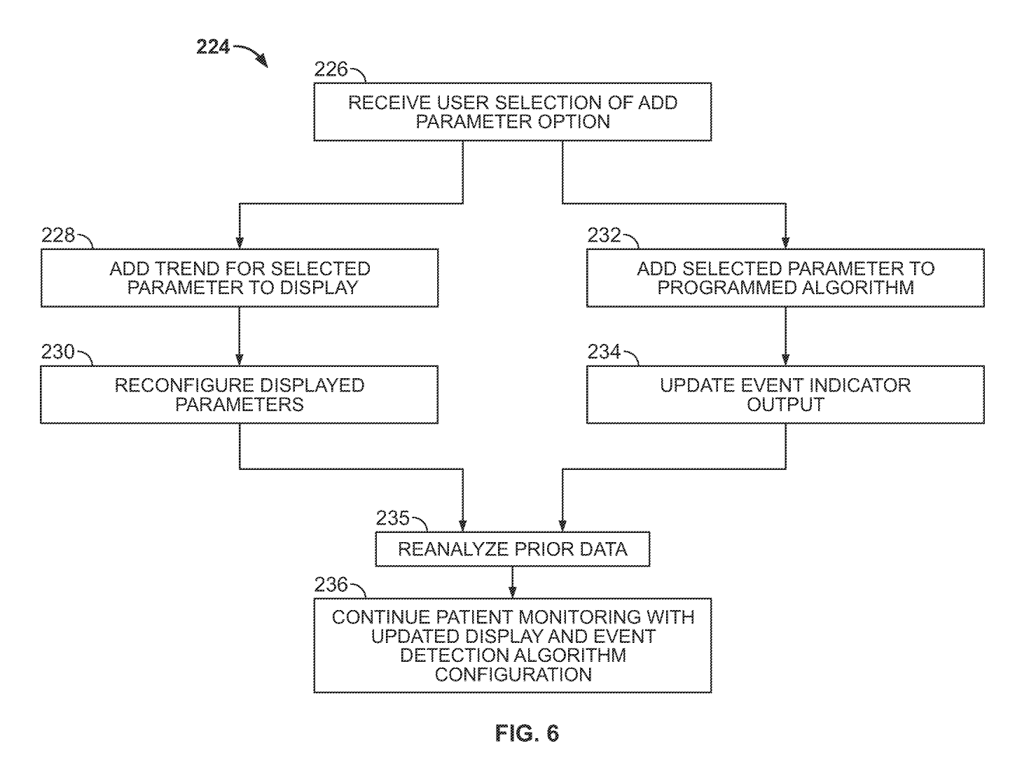

While FIGS. 3-5 show the removal of a monitored EEG parameter, extracted EEG signal parameters may also be added to the monitoring algorithm and the monitoring screens after a user selection. The method and display screens shown in FIGS. 6-8 illustrate one embodiment of adding a parameter to a display and a monitoring algorithm in response to a user's selection. The system and screens shown may, for example, be a response to a user's selection of a parameter from the parameter menu 122 discussed above and shown in display screen 100 of FIG. 1. In particular, FIGS. 6-8 show a response to a user's selection of the breathing parameter 136 from the menu 122.

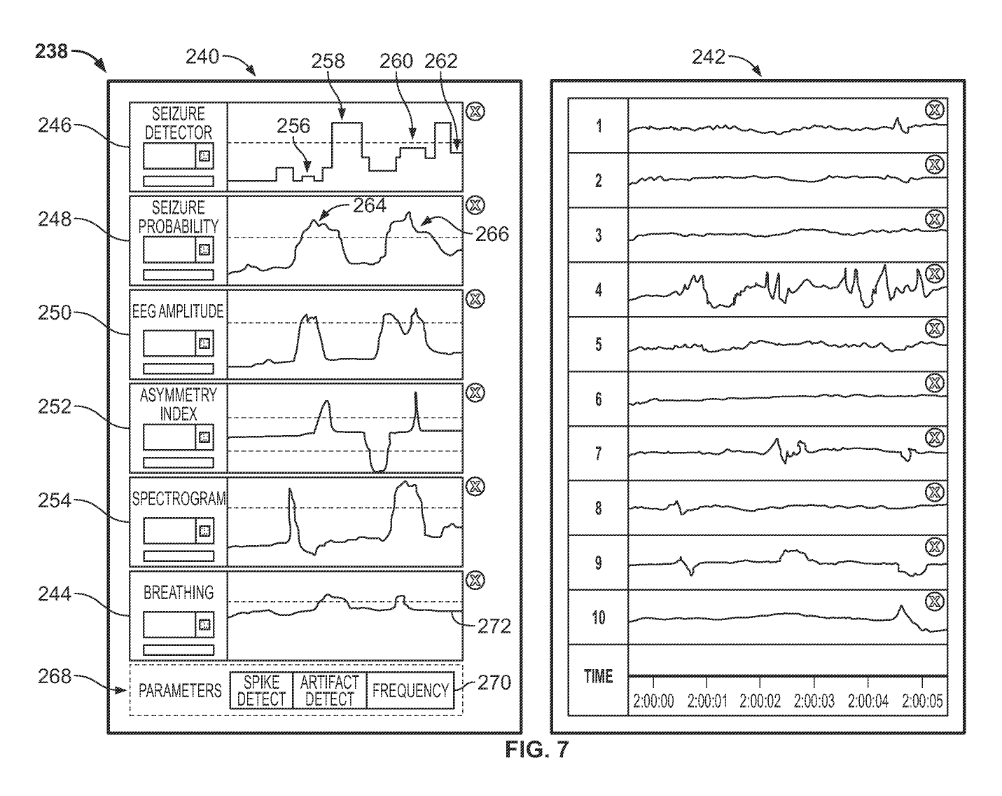

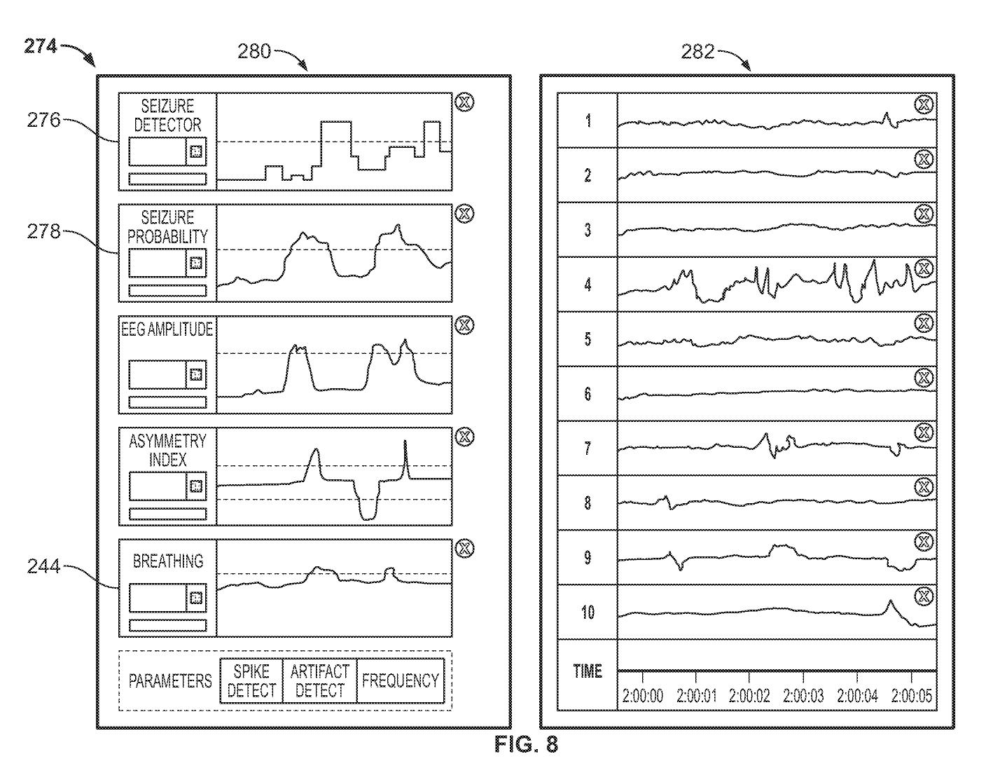

The method of updating the display and algorithms when the breathing parameter 136 is selected from the screen shown in FIG. 1 is shown in flowchart 224 of FIG. 6. The method begins when the user's selection of a parameter to add is received at step 226. As with the process of removing a parameter discussed above and shown in FIG. 3, the updates after the user's selection to add a parameter to monitoring follows two parallel paths. The two parallel paths serve to update both the display presented to the physician and the underlying monitoring algorithm in response to the user's single selection of a parameter to add from the monitoring window. The first updating process begins at step 228 when the selected parameter trend is added to the parameter window presented to the physician. As is shown in FIGS. 7 and 8, adding this trend to the display parameter window may include either adding the trend in place of one of the other currently displayed trends or may include displaying the new trend in addition to those already displayed. The displayed trends may be resized, rescaled or shifted around the display screen in response to the addition of the trend at step 230.

The parallel updating to the monitoring algorithms begins at step 232 when the data corresponding to the selected parameter is added to the monitoring algorithm. Adding the data to the algorithm may include reprogramming the algorithm or adjusting the weight of various parameters relative to each other to add the data to the calculation. For example, the weights of all non-monitored parameters may be set to zero during ongoing monitoring, and in response to the selection of a parameter, the weight for that parameter may be increased from zero to begin to use it in the event indicator calculations. As the parameter is added, the corresponding weights of other parameters in the algorithm may be updated as needed to account for the new parameter.

Following the algorithm updates, the event indicator trend is updated at step 234 to account for the reprogrammed algorithm updated at step 232. The event indicator trend updated at step 234 may be either retroactively updated or only changed going forward on the fly from the time of receipt of the user's selection at step 226. If the event indicator and trends are updated retroactively, the prior data is reanalyzed at step 235 to calculate the new trends for the past data. If the data is only updated going forward, and not retroactively, then step 235 is bypassed, and the changes take effect for only future data. Once both the display of parameters and the programmed algorithm are updated, patient monitoring continues at step 236 with the updated configuration.

FIGS. 7 and 8 show two display screens that may result from the updating performed by the monitoring system in response to the addition of a parameter in the method 224 of FIG. 6. FIG. 7 shows a display screen 238 that includes a parameter window 240 and an EEG sensor signal window 242. As with the displays in FIGS. 4 and 5, the EEG sensor signal window 242 is not changed from the window 104 in FIG. 1 because the received user selection to add a parameter does not affect the received EEG signals that are processed by the monitoring system. The parameter window 240 has, however, been updated from the parameter window 102 of FIG. 1 to account for the addition of the selected breathing parameter 244. As shown in the parameter window 240, the parameter trend 244 now displays the trend data 272 of a breathing parameter, for example a measured patient respiration rate, in the parameter window 240. The parameter menu 268 has also been updated to remove the breathing parameter option as it is now included in the displayed parameter window 240. The breathing parameter 244 has been replaced in the menu 268 by a new frequency parameter 270 that a user may subsequently select to add to the monitoring display 238.

To allow for the addition of the breathing parameter 244 to the display screen 238, the seizure detector and seizure probability trends 246 and 248, as well as the EEG amplitude parameter 250, asymmetry index parameter 252 and spectrogram parameter 254, can be scaled and resized to make room in the window 240 for the breathing parameter 244. Each of the extracted parameters 250, 252 and 254 display identical trends to the trends shown in FIG. 1 because the selection of a new parameter to the monitoring algorithm does not affect the extraction of these parameters from the EEG signals shown in signal window 242.

The calculated outputs shown in the seizure detector trend 246 and the seizure probability trend 248 have, however, changed relative to those shown in FIG. 1 as a result of the newly added parameter. In particular, the portions of the detector trend 246 indicated by pointers 256, 258, 250 and 262 exhibit a different shape relative to the corresponding portions of the trend 118 shown in FIG. 1 and discussed above. Likewise, the seizure probability trend 248 exhibits different shapes at peaks 264 and 266 relative to the corresponding portions of the original probability trend 120 shown in FIG. 1. As discussed with the removal of a parameter, the event indicator trends 246 and 248 may not be retroactively changed and may instead only show updated algorithm calculations going forward from the selection of a parameter to add. For the purpose of illustration, however, the changes are applied retroactively in the embodiment described in FIG. 7 to highlight the effect of the user selection of the breathing parameter 244 on the ongoing monitoring configuration.

Rather than resizing and scaling the output parameters shown in window 240, one or more of these parameters may be removed from the window to allow for the addition of the breathing parameter 244 without crowding the physician's display. This approach may be advantageous for embodiments in which a large number of parameters are used and are presented in the physician's display to assist the physician in tracking important parameters. An updated display screen 274 is shown in FIG. 8 for such an embodiment.

In the display screen 274, the parameter window 280 is updated to remove the spectrogram parameter 254 and replace that parameter with the selected breathing parameter 244. The other data displayed in the EEG sensor window 282 and extracted parameters shown in the parameter window 280 remain unchanged from the display screen 238 of FIG. 7 as the spectrogram parameter, though removed from the window 280, is still processed in the underlying monitoring algorithm the same as if it were still displayed to the physician. As a result, the seizure detector trend 276 and the seizure probability trend 278 are identical to the corresponding trends 246 and 248 of display screen 238.

FIGS. 3-8 illustrate options that are provided to a user for selecting parameters that are added or subtracted from the processing and monitoring algorithm used to determine event indicators. In addition to completely adding or removing these parameters from the underlying algorithms, the systems and methods described herein may also allow a user to adjust the process by which those parameters are combined to determine the event indicators. For example, the relative weighting of selected parameters may be changed, or the thresholds applied to each parameter may be adjusted in response to a physician's observations of the ongoing monitoring signals. These adjustments include changing individual thresholds for EEG channels or individual extracted parameters that are used in the detection algorithm. For example, a user may adjust a displayed threshold directly on the display screen, such as threshold 142 shown in FIG. 1. This adjustment may include clicking on the threshold 142 and dragging it up or down, or the threshold may be changed from an entry in the adjustment option box 138c. The adjustment may also include changing the particular EEG channels that are processed for a particular extracted parameter, for example by selecting individual EEG channels from a drop-down menu in the option box 138c of FIG. 1. Any number of other suitable individual parameter adjustments may be applied, and the examples discussed herein are illustrative and non limiting.

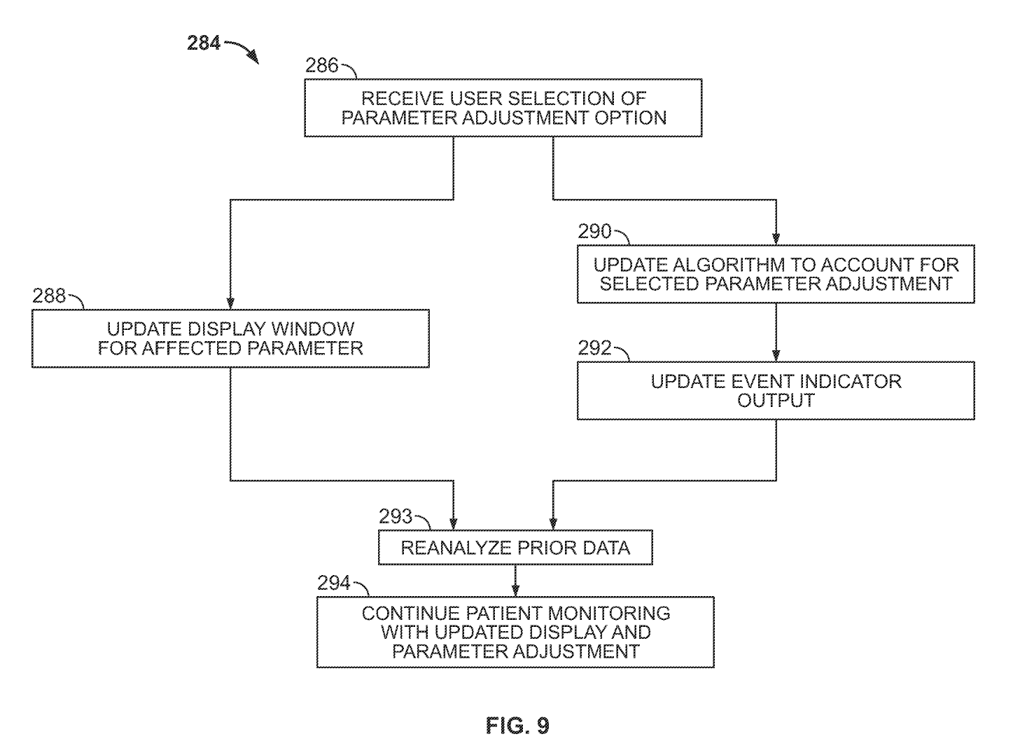

FIGS. 9-11 illustrate one approach for responding to a user selection of an individual parameter adjustment option. As with the approaches shown in FIGS. 3-8, the adjustments made in FIGS. 9-11 illustrate the flexibility of the systems and methods described herein to allow a physician to change both the display data and the underlying processing algorithm processing used for patient monitoring.

The process shown in the flowchart 284 in FIG. 9 begins when a user selection of a parameter adjustment option is received at step 286. The parameter adjustment option that is received at step 286 may be any of the options discussed herein or any other suitable options, but will be described as a selection of a change in the threshold 142 on the display 100 of FIG. 1 for the purposes of illustration. As with the adjustments made when parameters are added or removed from monitoring, the method in FIG. 9 performs a parallel update of both the display provided to the physician and the underlying monitoring algorithm in response to the single selection received at step 286.

The first portion of the parallel update occurs at step 288 when the display window is updated for the parameter affected by the selected adjustment. The update at step 288 in the method 284 includes changing the position of the threshold 142 shown in FIG. 1 to respond to the user's selection. In cases where other options are selected, the affected parameter may be changed to show an updated trend or another updated aspect of the parameter in response to the selection. For example, if different EEG monitoring channels were selected from the option box 138c of FIG. 1, the trend of the EEG amplitude parameter 112 may be updated to account for the newly-monitored EEG channels and corresponding data.

The second arm of the parallel update begins at step 290 in which the underlying monitoring algorithm is reprogrammed to account for the selected parameter adjustment. In the case of a change in the threshold 142, the update occurring at step 290 is a change in the algorithm variable that is the threshold value to which data from the EEG amplitude parameter is compared. In other examples, this algorithm update may include changing weighting of different EEG channels or changing the processing of a given extracted parameter in response to the type of adjustment option that is received at step 286. After the algorithm is adjusted at step 290, the event indicator trend is updated at step 292 to account for the parameter adjustment. The change at step 292 may include a retroactive update of the displayed event indicators or may only change the event indicators going forward from the time at which the selection is received at step 286. If the event indicators are updated retroactively, the prior data is reanalyzed at step 293 to update the past trends. If the adjustments are only used going forward, step 293 is bypassed, and the changes take effect only for future data. After the parallel updates to the display and the programmed algorithm, patient monitoring continues at step 294 with the adjusted settings requested by the user at step 286.

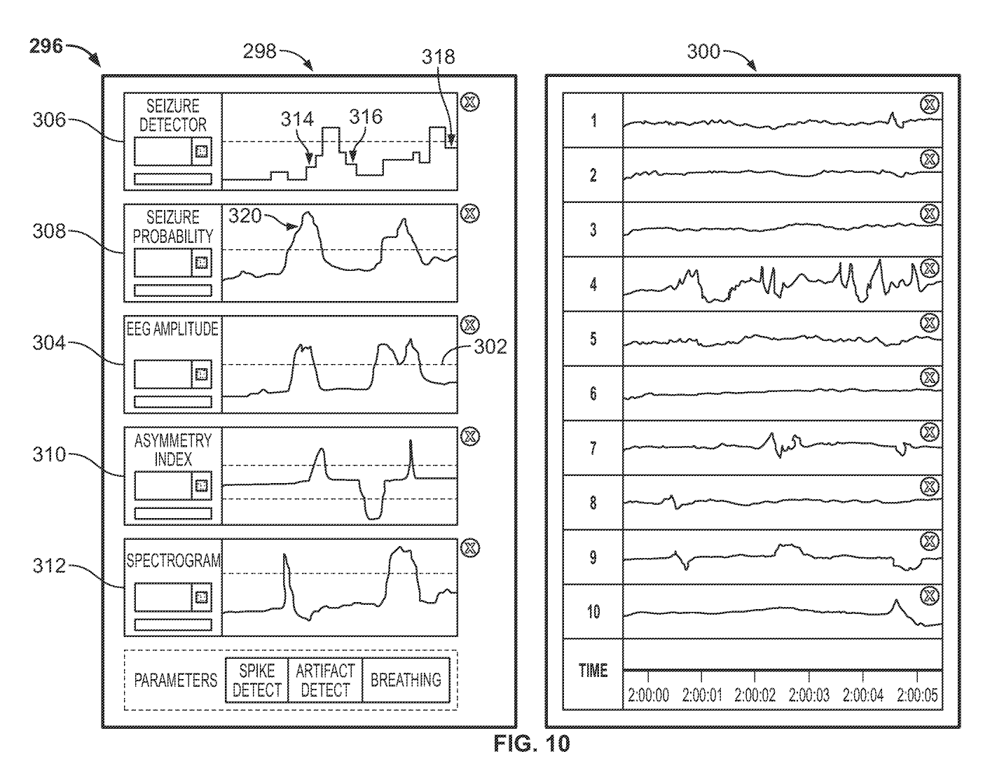

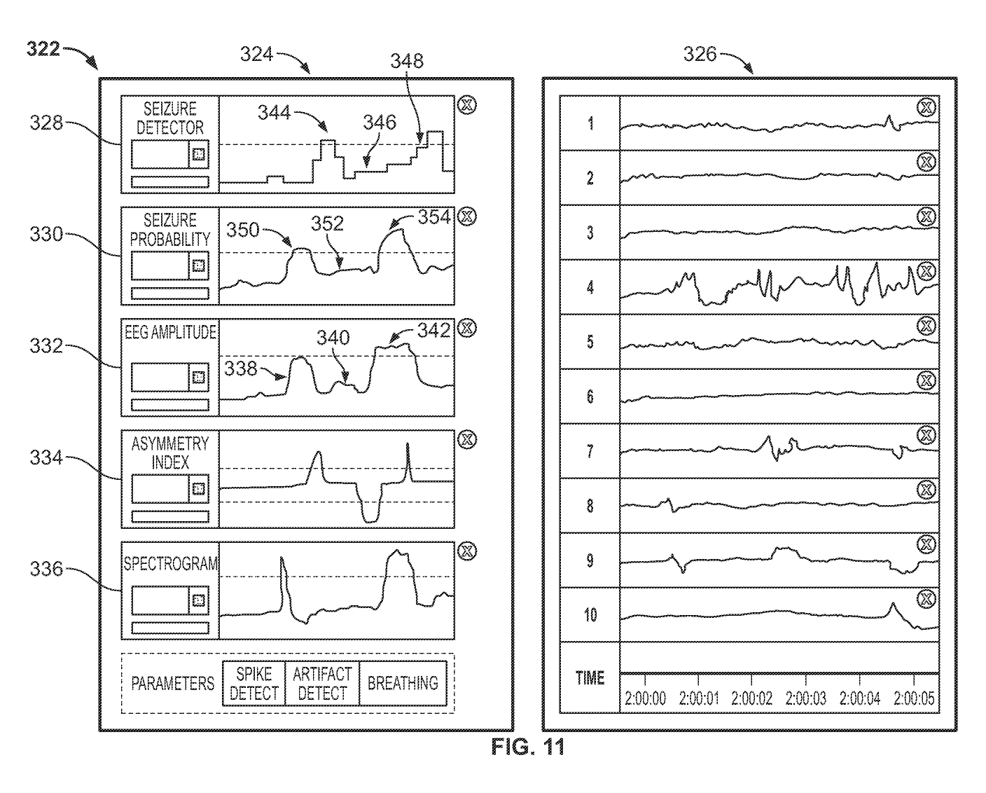

The changes to the physician's display and to the underlying monitoring algorithm made in the method shown in FIG. 9 is illustrated in the display screens shown in FIGS. 10 and 11. The display screen 296, shown in FIG. 10, includes a parameter window 298 and an EEG sensor signal window 300. As shown, the EEG sensor signal window 300 is identical to the corresponding window 104 in FIG. 1 because the parameter adjustment option does not affect the received and processed raw data from the EEG sensors.

The parameter window 298 in display screen 296 is updated to account for the user's requested change to the threshold 302 for the EEG amplitude parameter 304. In particular, the new threshold 302 for EEG amplitude 304 is lower than the corresponding threshold 142 shown in FIG. 1. This change affects the value at which the EEG amplitude parameter 304 is determined to be indicative of either the onset or risk of a seizure. Lowering that threshold to the level of threshold 302 may effectively make the algorithm used to detect seizures more sensitive to increases in EEG amplitude.

The result of the change to the threshold 302 is a slight change in shape of the seizure detector trend 306 and the seizure probability trend 308. As shown, the portions of the trends indicated by pointers 314, 316 and 318 of the seizure detector trend 306 are slightly changed from the corresponding portions in the seizure detector trend 118 of FIG. 1. Likewise, the peak 320 of seizure probability trend 308 is slightly larger and wider than the corresponding peak 212 in the seizure probability trend 120 of FIG. 1. The changes to both the seizure detector trend 306 and seizure probability trend 308 are relatively minor compared to the trend in FIG. 1. This may result because the change to threshold 302 is only a small change from the corresponding threshold 142 from display screen 100. In some implementations, changes to a single parameter may have large implications, and the effects on the seizure detector and seizure probability graphs may be larger than those shown in FIG. 10. The asymmetry index parameter 310 and spectrogram parameter 312 are not changed relative to FIG. 1 because no adjustment is made to those parameters in response to only a change in the threshold of the EEG amplitude parameter 304.

Instead of a change to a threshold applied to a parameter, a different parameter adjustment may be requested by the physician. For example, instead of changing the threshold of the EEG amplitude parameter, a physician may instead select adjustment option 138c from the display screen 100 of FIG. 1 and make a change to the individual EEG channels that are processed and monitored for the EEG amplitude parameter. This change will not affect the threshold applied to the data but may affect the combined EEG amplitude parameter data when the EEG signals are processed. Display screen 322 of FIG. 11 shows an updated display when the EEG channels for the EEG amplitude parameter 332 are adjusted rather than changing the parameter's threshold. As shown in the display screen 322, the EEG sensor signal window 326 is not changed relative to the previous figures because the change affects only the EEG channels processed for the EEG amplitude parameter 332 and does not affect the full set of EEG data that is processed by the monitoring system.

The parameter window 324 in FIG. 11 is updated to account for the change in channels processed for the EEG amplitude parameter 332. For that parameter, portions of the trend indicated by pointers 338, 340 and 342 exhibit a different shape relative to those shown in the EEG amplitude parameter 304 in FIG. 10. This change in shape of the EEG amplitude parameter 332 is the result of a different set of EEG channel data being processed to extract this parameter in response to the physician's selection. While the EEG amplitude parameter 332 is changed, both the asymmetry index parameter 334 and the spectrogram parameter 336 are not changed because the physician's selection of channels was limited only to those channels processed to determine the EEG amplitude parameter 332.

Although the particular parameters combined in the monitoring algorithm are not changed by the user selection of channels for the EEG amplitude parameter 332, the EEG amplitude data that is processed in that algorithm is changed relative to the display shown in FIG. 1. As a result, the seizure detector trend 328 and the seizure probability trend 330 are updated relative to the corresponding detector trend 118 and probability trend 120 of FIG. 1. In particular, in seizure detector trend 328, the portions of the trend indicated by pointers 344, 346, and 348 exhibit a different shape as a result of the new EEG amplitude parameter 332 data. Likewise, the shape of the seizure probability trend 330 is updated at the portions indicated by pointers 350, 352, and 354 relative to the corresponding trend 120 of FIG. 1.

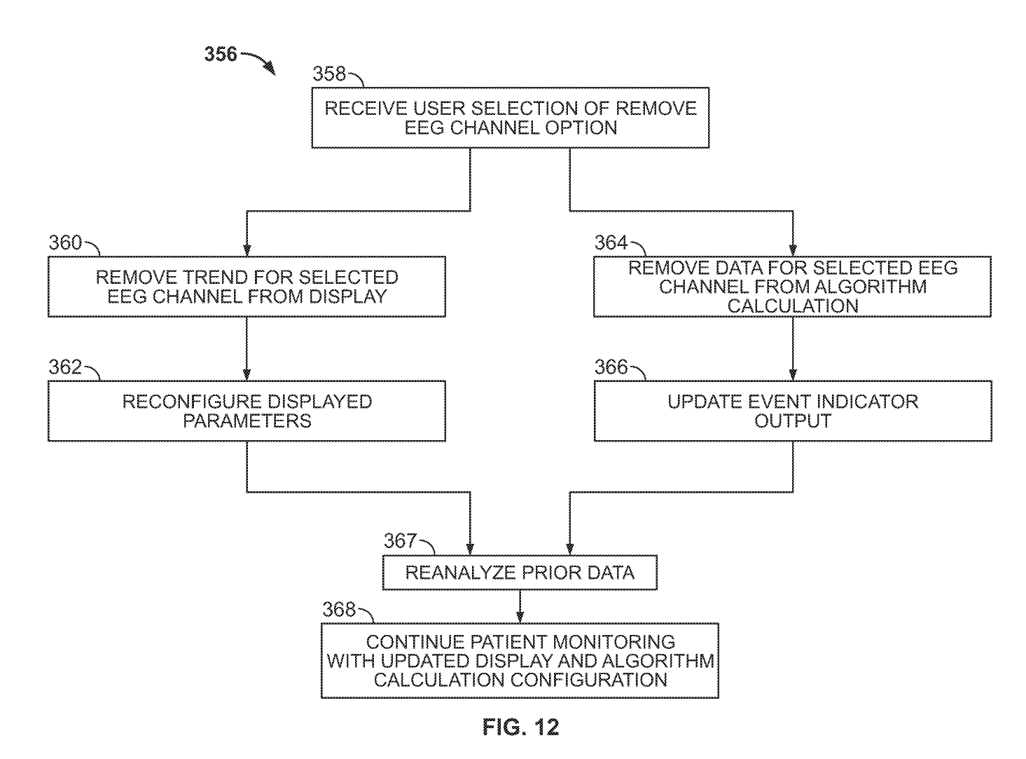

In addition to selecting particular EEG signal channels for a given extracted parameter, a user may also select EEG signal channels to be included or excluded from the full patient monitoring analysis. If, for example, a physician notices that an EEG data trend for a particular sensor is irregular and unreliable, the physician can select to remove data correspond to that channel from all parameters and event indicator calculations performed during patient monitoring. Such a selection causes the system to make another parallel update, first updating the physicians display not only to update the displayed parameters but also to update the display EEG sensor signals, and second updating the monitoring algorithm to exclude the noisy data that could compromise patient monitoring if it is processed. A method and display screens implementing this approach is shown in FIGS. 12-14 for the selection of an EEG channel to be excluded from monitoring, for example a selection of option 370 shown in FIG. 1 to remove data from EEG channel 7 from monitoring.

The method 356 shown in FIG. 12 begins at step 358 when a selection to remove and EEG channel, for example a selection of the remove option 370, is received. Similar to the approaches discussed above, the single selection triggers a parallel update to both the physician's display and to the underlying monitoring algorithm. In contrast to the previous approaches, the selection of option 370 affects not only the extracted parameters, but also affects the raw EEG signal data that is processed by the monitoring algorithms. As a result, the update to the physician's display affects not only the displayed parameters but also the displayed EEG signals. The display update begins at step 360, in which the trend corresponding to the selected EEG channel is removed from the physician's display screen.

The remaining EEG channels and the displayed parameters are then reconfigured and updated at step 362. In this step, the trend for the selected EEG channel is removed from the display screen, and the displayed parameters are updated to account for the removal of data from the selected channel in extracting the parameters from the raw EEG data. The removed EEG channel is then replaced with another monitored EEG channel signal, or the system may resize the remaining channels to fill the blank space without adding new channel data.

The parallel update to the underlying algorithm begins at step 364 in which the algorithm is reprogrammed to exclude data from the selected channel in calculating event indicators. Similar to the removal of a parameter from the algorithm, the exclusion of an EEG channel may be effected by weighting data from that channel to zero. At step 366, the reprogrammed algorithm is applied to update the event indicator trends based on the exclusion of the EEG data. If the EEG data exclusion is applied retroactively, past data is reanalyzed at step 367 to update the past trends. If the exclusion takes effect only going forward, step 367 is bypassed. After the algorithm and display updates are complete, patient monitoring continues at step 368 with the new configuration that excludes data from the selected EEG channel in ongoing monitoring.

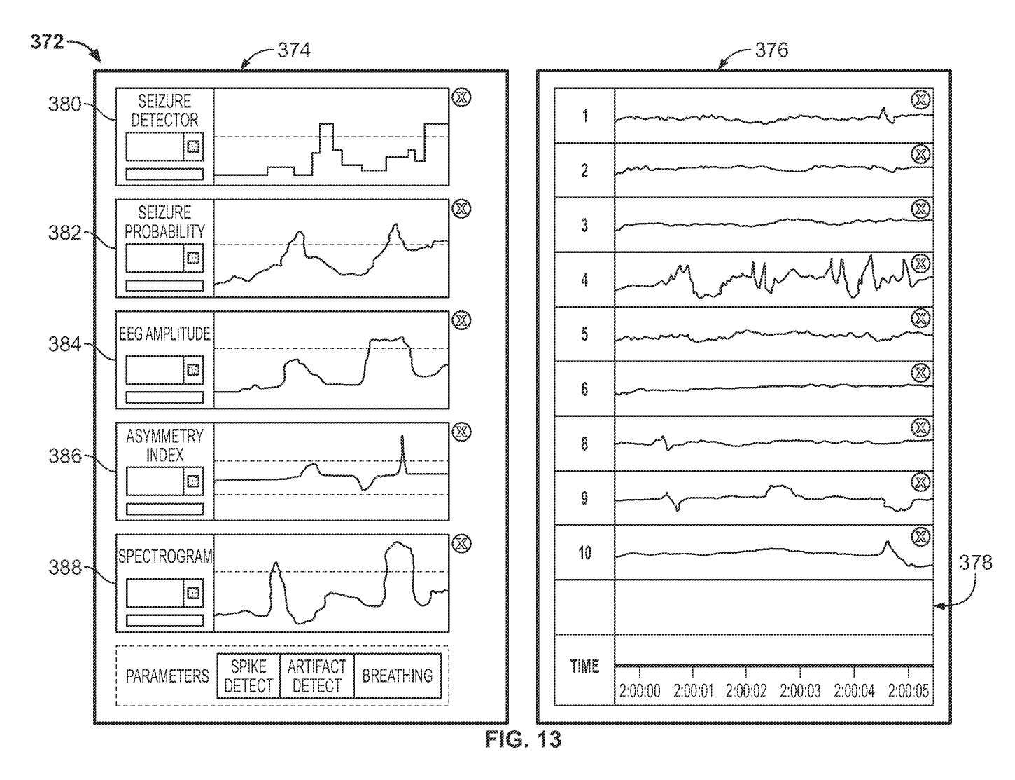

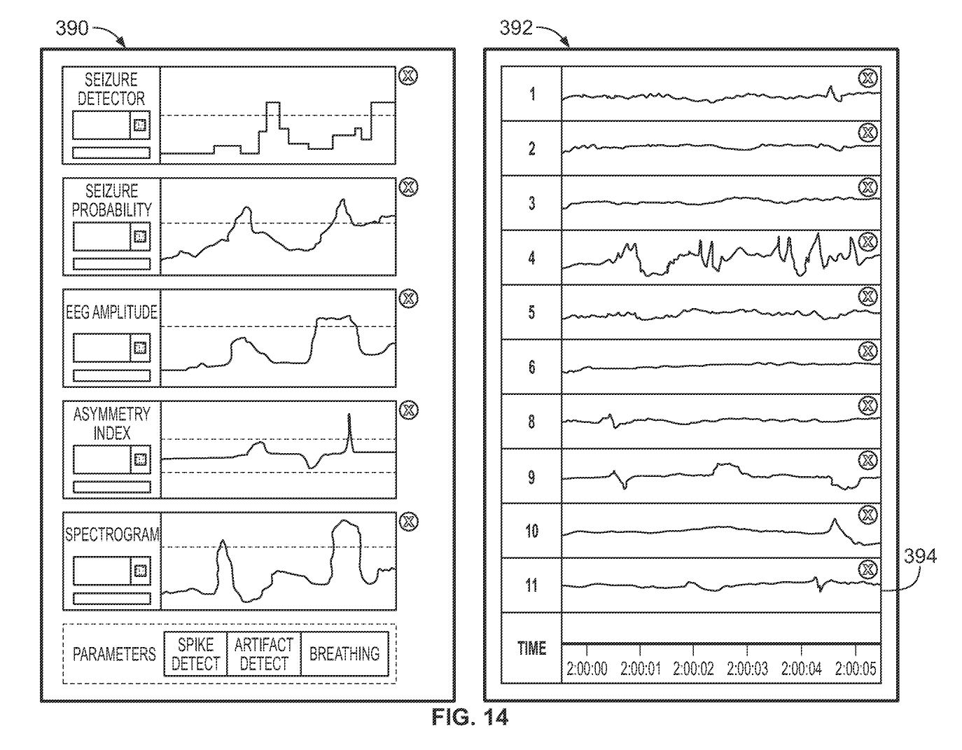

The effects of both the display and the algorithm updates are illustrated in the revised display screens shown in FIGS. 13 and 14. In FIG. 13, a display screen 372 includes an updated parameter window 374 and an updated EEG sensor signal window 376. The signal window 376 has been revised to remove the data trend from the removed EEG channel 7. With that channel removed, EEG channels 8-10 have been shifted upwards in the display, and a blank area 378 is left where channel 10 was previously displayed. This area 378 is blank in FIG. 13, but in alternative embodiments the space may be automatically replaced with another monitored EEG channel. For example, FIG. 14 shows an updated display screen 390 in which the EEG sensor signal window 392 is updated to remove EEG channel 7 and replace that channel automatically with the next channel, EEG channel 11 data shown by trend 394. In other embodiments, the user may be presented with an option to select a channel to replace the removed EEG data rather than selecting that channel automatically.

In the parameter window 374 of display screen 372, each of the displayed trends—seizure indicator trend 380, seizure probability trend 382, EEG amplitude parameter 384, asymmetry index parameter 386, and spectrogram parameter 388—are changed relative to display screen 100 of FIG. 1. Each trend is different because, in contrast to the selection of the channel option for a single parameter, the selection of the remove option 370 excludes that data channel across all parameters and event indicators in the monitoring process. As a result, there are changes in the trends for each parameter 384, 386, and 388, as well as for each event indicator 380 and 382. As with the displays discussed above, the display screen 372 retroactively updates the data trends to highlight the effect of the user's selection, but in other applications the trends may update only from the point at which the selection is made.

### Example Embodiments

A1. A method for monitoring EEG signals, comprising:
receiving an EEG sensor signal;
extracting a plurality of parameters from the received signal;
determining, with a processor, an event indicator from the plurality of extracted parameters;
generating a display screen that includes:
   the event indicator;
   the extracted plurality of parameters; and
   a user selectable option;
receiving a user selection of the option in the display screen; and
in response to the user selection, updating the extracted plurality of parameters in the display screen and updating an algorithm used to determine the event indicator.

A2. The method of A1, wherein the display screen includes a threshold displayed with each of the plurality of extracted parameters, the user selectable option comprising a request to change the displayed thresholds.

A3. The method of A2, wherein the request to change the displayed threshold comprises at least one of a numerical value entry, a menu of selectable thresholds, and an adjustable threshold cursor.

A4. The method of any of A1-A3, wherein the display screen includes an alert for each of the plurality of extracted parameters, each alert indicating whether a corresponding extracted parameter exceeds a threshold.

A5. The method of any of A1-A4, wherein the display screen includes a user selectable menu of available parameters.

A6. The method of A5, wherein the menu of available parameters includes only parameters that are not displayed on the display screen and not used in the event indicator determination.

A7. The method of A5, wherein the menu of available parameters includes all parameters that can be extracted from the EEG sensor signal.

A8. The method of A7, wherein the display screen includes a marker for a first set of extracted parameters that are used in the event indicator determination to differentiate the first set of extracted parameters from a second set of parameters that are not used in the event indicator determination.

A9. The method of any of A5-A8, wherein:
the user selection of an option comprises a selection of an unused parameter from the menu;
updating the extracted plurality of parameters in the display screen comprises generating a graph of a trend for the unused parameter in the display; and
updating the algorithm comprises reprogramming the algorithm to include the unused parameter in the event indicator determination.

A10. The method of any of A1-A9, wherein the received signal comprises data from a plurality of EEG channels, and the display screen includes a user selectable menu of the EEG channels.

A11. The method of A10, wherein:
the user selection of an option comprises a request to include or exclude an EEG channel during patient monitoring;
updating the extracted plurality of parameters in the display screen comprises graphing updated trends for the displayed parameters based on the inclusion or exclusion of the EEG channel; and
updating the algorithm comprises reprogramming the algorithm to include or exclude data for the selected EEG channel from at least one extracted parameter.

A12. The method of A11, wherein the request is a request to include or exclude data for the selected EEG channel from all of the extracted parameters.

A13. The method of A11, wherein the request is a request to include or exclude data for the selected EEG channel from one of the extracted parameters without affecting EEG channel data for additional extracted parameters.

A14. The method of any of A1-A13, wherein the algorithm comprises weighting factors associated with each of the plurality of extracted parameters.

A15. The method of A14, wherein the display screen includes user selectable options to change the weighting factors associated with the displayed extracted parameters.

A16. The method of any of A1-A15, wherein the event indicator includes at least one of an alert that an event has happened, a warning that an event will happen, a percentage estimate of the chance an event will happen, and a binary indication of whether an event is currently happening.

A17. The method of any of A1-A16, wherein the event indicator is a seizure indicator.

B1. A system for monitoring EEG signals, comprising:
an EEG sensor;
a monitor configured to receive an EEG signal from the EEG sensor, the monitor comprising a processor configured to:
   generate a display screen that includes an event indicator, a plurality of parameters extracted from the received signal, and a user-selectable option;
   receive a user selection of the option in the display screen; and
   in response to the user selection, update the extracted plurality of parameters in the display screen and update an algorithm used to determine the event indicator.

B2. The system of B1, wherein the processor is configured to carry out any of the methods of A1-A17.

B3. The system of B1, wherein the monitor comprises communications circuitry.

B4. The system of B3, wherein the communications circuitry is configured to transmit the generated display screen to a display device.

B5. The system of B4, wherein the communications circuitry is configured to receive the user selection of the option from the display device.

B6. The system of B3, wherein the communications circuitry is configured to send commands to the EEG sensor.

B7. The system of any of B1-B6, wherein the monitor comprises a user interface.

B8. The system of B7, wherein the monitor is configured to receive the user selection of the option from the user interface.

B9. The system of B8, wherein the selected option is displayed on a display device in communication with the monitor.

C1. A system for monitoring EEG signals, comprising:
means for receiving an EEG sensor signal;
means for extracting a plurality of parameters from the received signal;
means for determining an event indicator from the plurality of extracted parameters;
means for generating a display screen that includes:
   the event indicator;
   the extracted plurality of parameters; and
   a user selectable option;
means for receiving a user selection of the option in the display screen; and
means for updating the extracted plurality of parameters in the display screen in response to the user selection; and
means for updating an algorithm used to determine the event indicator in response to the user selection.

C2. The system of C1, wherein the display screen includes:
a threshold displayed with each of the plurality of extracted parameters; and
user selectable options to change the displayed thresholds.

C3. The system of C2, wherein the user selectable options comprise at least one of a numerical value entry, a menu of selectable thresholds, and an adjustable threshold cursor.

C4. The system of any of C1-C3, wherein the display screen includes an alert for each of the plurality of extracted parameters, each alert indicating whether a corresponding extracted parameter exceeds a threshold.

C5. The system of any of C1-C4, wherein the display screen includes a user selectable menu of available parameters.

C6. The system of C5, wherein the menu of available parameters includes only parameters that are not displayed on the display screen and not used in the event indicator determination.

C7. The system of C5, wherein the menu of available parameters includes all parameters that can be extracted from the EEG sensor signal.

C8. The system of C7, wherein the display screen includes a marker for a first set of extracted parameters that are used in the event indicator determination to differentiate the first set of extracted parameters from a second set of parameters that are not used in the event indicator determination.

C9. The system of any of C5-C8, wherein the user selection of an option comprises a selection of an unused parameter from the menu, the system further comprising:
means for generating a graph of a trend for the unused parameter in the display; and
means for reprogramming the algorithm to include the unused parameter in the event indicator determination.

C10. The system of any of C1-C9, wherein the received signal comprises data from a plurality of EEG channels, and the display screen includes a user selectable menu of the EEG channels.

C11. The system of C10, wherein the user selection of an option comprises a request to include or exclude an EEG channel during patient monitoring, the system further comprising:
means for graphing updated trends for the displayed parameters based on the inclusion or exclusion of the EEG channel; and
means for reprogramming the algorithm to include or exclude data for the selected EEG channel from at least one extracted parameter.

C12. The system of C11, wherein the request is a request to include or exclude data for the selected EEG channel from all of the extracted parameters.

C13. The system of C11, wherein the request is a request to include or exclude data for the selected EEG channel from one of the extracted parameters without affecting EEG channel data for additional extracted parameters.

C14. The system of any of C1-C13, wherein the algorithm comprises weighting factors associated with each of the plurality of extracted parameters.

C15. The system of C14, wherein the display screen includes user selectable options to change the weighting factors associated with the displayed extracted parameters.

C16. The system of any of C1-C15, wherein the event indicator includes at least one of an alert that an event has happened, a warning that an event will happen, a percentage estimate of the chance an event will happen, and a binary indication of whether an event is currently happening.

C17. The system of any of C1-C16, wherein the event indicator is a seizure indicator.

The foregoing is merely illustrative of the principles of the disclosure, and the systems, devices, and methods can be practiced by other than the described embodiments, which are presented for purposes of illustration and not of limitation. It is to be understood that the systems, devices, and methods disclosed herein, while shown for use in wound monitoring approaches using wound dressing having color pH indicators, user devices, and servers, may be applied to systems, devices, and methods to be used in other approaches for wound monitoring using pH tracking or tracking of other wound indicators using color bandages.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

What is claimed is:

1. A method for monitoring EEG signals, comprising:
receiving an EEG sensor signal;
extracting a plurality of parameters from the received signal;
determining, with a processor, an event indicator from the plurality of extracted parameters;
generating a display screen that includes:
the event indicator;
the extracted plurality of parameters; and
a user selectable option;
receiving a user selection of the option in the display screen; and
in response to the user selection, updating the extracted plurality of parameters in the display screen and updating an algorithm used to determine the event indicator.

2. The method of claim 1, wherein the display screen includes a threshold displayed with each of the plurality of extracted parameters, the user selectable option comprising a request to change the displayed thresholds.

3. The method of claim 2, wherein the request to change the displayed threshold comprises at least one of a numerical value entry, a menu of selectable thresholds, and an adjustable threshold cursor.

4. The method of claim 1, wherein the display screen includes an alert for each of the plurality of extracted parameters, each alert indicating whether a corresponding extracted parameter exceeds a threshold.

5. The method of claim 1, wherein the display screen includes a user selectable menu of available parameters.

6. The method of claim 5, wherein the menu of available parameters includes only parameters that are not displayed on the display screen and not used in the event indicator determination.

7. The method of claim 5, wherein the menu of available parameters includes all parameters that can be extracted from the EEG sensor signal.

8. The method of claim 7, wherein the display screen includes a marker for a first set of extracted parameters that are used in the event indicator determination to differentiate the first set of extracted parameters from a second set of parameters that are not used in the event indicator determination.

9. The method of any of claim 5, wherein:
the user selection of an option comprises a selection of an unused parameter from the menu;
updating the extracted plurality of parameters in the display screen comprises generating a graph of a trend for the unused parameter in the display; and
updating the algorithm comprises reprogramming the algorithm to include the unused parameter in the event indicator determination.

10. The method of claim 1, wherein the received signal comprises data from a plurality of EEG channels, and the display screen includes a user selectable menu of the EEG channels.

11. The method of claim 10, wherein:
the user selection of an option comprises a request to include or exclude an EEG channel during patient monitoring;
updating the extracted plurality of parameters in the display screen comprises graphing updated trends for the displayed parameters based on the inclusion or exclusion of the EEG channel; and
updating the algorithm comprises reprogramming the algorithm to include or exclude data for the selected EEG channel from at least one extracted parameter.

12. The method of claim 11, wherein the request is a request to include or exclude data for the selected EEG channel from all of the extracted parameters.

13. The method of claim 11, wherein the request is a request to include or exclude data for the selected EEG channel from one of the extracted parameters without affecting EEG channel data for additional extracted parameters.

14. The method of any of claim 1, wherein the algorithm comprises weighting factors associated with each of the plurality of extracted parameters.

15. The method of claim 14, wherein the display screen includes user selectable options to change the weighting factors associated with the displayed extracted parameters.

16. The method of any of claim 1, wherein the event indicator includes at least one of an alert that an event has happened, a warning that an event will happen, a percentage estimate of the chance an event will happen, and a binary indication of whether an event is currently happening.

17. The method of any of claim 1, wherein the event indicator is a seizure indicator.

18. A system for monitoring EEG signals, comprising:
an EEG sensor;
a monitor configured to receive an EEG signal from the EEG sensor, the monitor comprising a control processor configured to:
generate a display screen that includes an event indicator, a plurality of parameters extracted from the received signal, and a user-selectable option;
receive a user selection of the option in the display screen; and
in response to the user selection, update the extracted plurality of parameters in the display screen and update an algorithm used to determine the event indicator.

19. The system of claim 18, wherein the monitor comprises a user interface.

20. The system of claim 19, wherein the monitor is configured to receive the user selection of the option from the user interface.
